(19) United States
(12) Patent Application Publication
(10) Pub. No.: US 2002/0039785 A1
(43) Pub. Date: Apr. 4, 2002

(54) Cell-culture vessel

(76) Inventors: Kirk S. Schroeder, Ann Arbor, MI (US); Brad D. Neagle, Ann Arbor, MI (US)

Correspondence Address: John G. Posa, Gifford, Krass, Groh, Suite 400, 280 N. Old Woodward Ave., Brimingham, MI, 48009, US

(21) Appl. No.: 09/948,863

(22) Filed: Sep. 7, 2001

Related U.S. Application Data
(60) Provisional application No. 60/237,535, filed on Oct. 4, 2000.

(51) Int. Cl. C12M 1/24

(52) U.S. Cl. 435/304.3; 435/297.5

(57) ABSTRACT

An improved tissue culture flask vessel includes an access port that is more amenable to an automated removal and delivery of fluid. A second lid is located on the top side of the flask toward the conventional side cap location such that a slight tilt of the flask locates the fluid in the front corner of the flask which can easily be accessed by a top-down pipette of much shorter length, on the order of two inches. This greatly reduces the angular tolerances for pipette access as well as significantly reduces the amount of tilt manipulation required to locate the fluid for pipette access. The lid access is preferably recessed on the flask, so that the flasks can also be stacked on top of one another as is common practice in storage. The existing lid is preferably preserved to maintain proper gas exchange. To take advantage of the fact that the cells do not require much height, alternative embodiments feature a multi-layer surface within a given flask. In these cases the fluid is re-distributed to the various interior surface layers by a unique manipulation of the flask. These designs allow for two or more surfaces to be accessed within the same flask by a single top access port.

### REFERENCE TO RELATED APPLICATIONS

This application claims priority from provisional patent application Serial No. 60/237,535, filed Oct. 4, 2000, the entire contents of which are incorporated herein by reference.

### FIELD OF THE INVENTION

This invention is generally directed to improving the accessibility of aseptic fluids in cell culture. Specifically, the invention resides a tissue culture flask conducive to automated cell culture which provides an easily accessible port for fluid removal and addition.

### BACKGROUND OF THE INVENTION

The growth and maintenance of cell cultures is a common practice in academia, biotech, and pharmaceutical companies. Following various protocols, cells are allowed to reproduce in physiologically favorable vessels until they reach a given density. At this density the cells are then “thinned” and re-seeded into new culture vessels whereby they begin to reproduce again. The general process of cell culture therefore relies on the removal and addition of various types of fluids to the vessels at specified intervals during the culture process.

Usually the vessels are optically clear such that the cells can be imaged using a microscope. The purpose of the vessels is multifold: 1) to provide an aseptic environment; 2) to contain the required growth media solution; and 3) to allow for the exchange of gases (e.g. CO₂ or O₂) necessary for maintaining proper metabolism and pH.

Many types of vessels have been developed to allow for the continuous culture of various cell lines. These vessels take many shapes and sizes and are comprised of either glass or plastic. The most common vessels are referred to as T-flasks and come in various sizes, all with the same basic shape depicted in FIG. 1. They are fabricated from molded plastic, typically polystyrene, and are sterilized by gamma irradiation before packaging. The interior surface is often coated and/or treated on the bottom inner surface of the flask to promote the growth of adherent cell lines. Surface treatments include chemical alterations, such as the application of a chemical coating, or they may involve surface modifications such as with the use of electrical surface treatment (EST) to change the composite charge of the base material.

Since the cells require only a small layer of fluid above them, typically on the order of a few millimeters, the flasks tend to be short in height as compared to length and width. Quite often the size of flask is denoted by the growth surface area, for example a T-75 defines a 75-cm2 growth surface area flask. During culture, the flasks are set flat in a humidified and temperature-controlled environment. With adherent cell lines, cells are only grown on the bottom interior surface of the flask.

Referring to FIG. 2, accessibility to the fluid 2 in the flask 4 is provided by the removal of a lid 6 on one end of the flask. This lid is often fitted with a filter 8 providing gas exchange, but limiting the passage of particulates (e.g. air-borne pollen or spores). In order to remove the fluid, the flask is manually tipped up away from the lid such that the fluid flows into the opposite bottom corner of the flask. An operator then lowers a hand-held pipette 10 attached to a hand-held pump mechanism 12 into the bottom of the flask and aspirates manually. Fluid is added in a similar fashion. Due to the size of some of the larger culture flasks, the pipettes required to reach the bottom of the flasks can be as long as 13 inches. The length of the pipette in combination with the tipping of the flask demands a degree of manual manipulation that is possible for a human but difficult to achieve in an automated robotic system.

Some flask designs incorporate more than one access port, but these products are only offered in conjunction with a complex partitioning arrangement for isolated cell growth which is not conducive to automated processes. In particular, the “CELLine” series of flasks from Integra Biosciences (Ijamsville, Md.) use a smaller cell cultivation chamber (5/15 ml) combined with a larger nutrient supply chamber (350/1000 ml). The cell compartment is separated by an upper semi-permeable membrane from the basal medium compartment. Nutrients and other small molecules pass across the semi-permeable membrane into and out of the cell compartment. The cells settle upon the bottom of the cell compartment atop of a gas exchange surface, across which oxygen and carbon dioxide rapidly diffuse. The cultivation chamber is accessed through a separate cap located on the top and rear area of the flask. When liquid is added or removed from the cell compartment, a separate cap associated with the nutrient compartment must first be loosened to prevent air lock. The nutrient compartment cap must then be tightened before placing the flask in the incubator. Shorter, 25 ml plastic serological pipettes may be used for cell compartment manipulations.

A multi-compartmentalized flask design of this type is shown in U.S. Pat. No. 5,693,537. According to one embodiment, two compartments reside one above the other and are in communication with a membrane selectively permeable to a specific class of molecules. The upper compartment houses basal medium and the lower compartment houses cells and cell culture medium. Selected nutrients and waste products are permitted to move freely between the basal medium and the cell culture medium. The upper basal medium compartment and the lower cell culture compartment are configured to allow pipette access. According to a different embodiment of the invention, oxygen tension within the cell culture compartment is controlled independent of ambient conditions by adding a third compartment that utilizes a variable level of liquid to alter oxygen tension.

U.S. Pat. No. 5,672,505 solves problems associated with tissue culture bottle access through the provision of an insert including an adapter for mounting a suspending arm. When the adapter is placed in the neck of the bottle, a specimen in the holder is suspended within the bottle. The neck adapter may also be incorporated into a cap for the bottle. The insert may include a vessel to be used for addition of substances or withdrawal of substances to or from a tissue culture without substantially disturbing growing cells, and is generally applicable to standard tissue culture bottles and other laboratory equipment.

To ensure that the culture medium can be withdrawn and replaced without manual handling and preferably without tilting so that the correct operational orientation of the vessel can be readily determined, the U.S. Pat. No. 6,150,159 discloses a vessel having at least one drainage channel which extends radially relative to the elongate axis of the vessel. This ensures that the great majority of the liquid nutrient can be efficiently drained out. Preferably the drainage channel(s) extends circumferentially for up to 5% or 9 degrees of the circumference of the cell culture vessel. This distance gives optimum drainage while ensuring that the optimum number of cells can be grown.

### SUMMARY OF THE INVENTION

This invention improves upon the prior art by providing a tissue culture flask vessel with an access port that is more amenable to an automated removal and delivery of fluid. The preferred embodiment uses the same basic T-flask configuration but further incorporates a second lid located on the top side of the flask toward the conventional side cap location. A slight tilt of the flask accordingly locates the fluid in the front corner of the flask which can easily be accessed by a top-down pipette of much shorter length, on the order of two inches. This greatly reduces the angular tolerances for pipette access as well as significantly reduces the amount of tilt manipulation required to locate the fluid for pipette access. All of these factors greatly increase the robustness of an automated approach to cell culture.

In the preferred embodiment the lid access is also recessed on the flask, so that the flasks can also be stacked on top of one another as is common practice in storage. The existing lid is preferably preserved to maintain proper gas exchange. To take advantage of the fact that the cells do not require much height, alternative embodiments feature a multi-layer surface within a given flask. In these cases the fluid is re-distributed to the various interior surface layers by a unique manipulation of the flask. These designs allow for two or more surfaces to be accessed within the same flask by a single top access port.

### BRIEF DESCRIPTION OF THE DRAWINGS

FIG. 1 is a drawing of a common prior-art T-flask, which is commercially available in various sizes;

FIG. 2 shows how the flask of FIG. 1 is accessed through the removal of a lid on one end of the flask, requiring pipettes as long as 13 inches;

FIG. 3A is a drawing which depicts a tissue culture flask vessel according to the invention having an access port which is more amenable to an automated removal and delivery of fluid;

FIG. 3B illustrates a robotic manipulation of the Flask of FIG. 3A;

FIG. 4A is a drawing of a two-level flask according to the invention illustrating liquid being added to or drawn from the upper level; and

FIG. 4B is a drawing of the two-level flask shown in FIG. 4A with liquid being added to or drawn from the lower level.

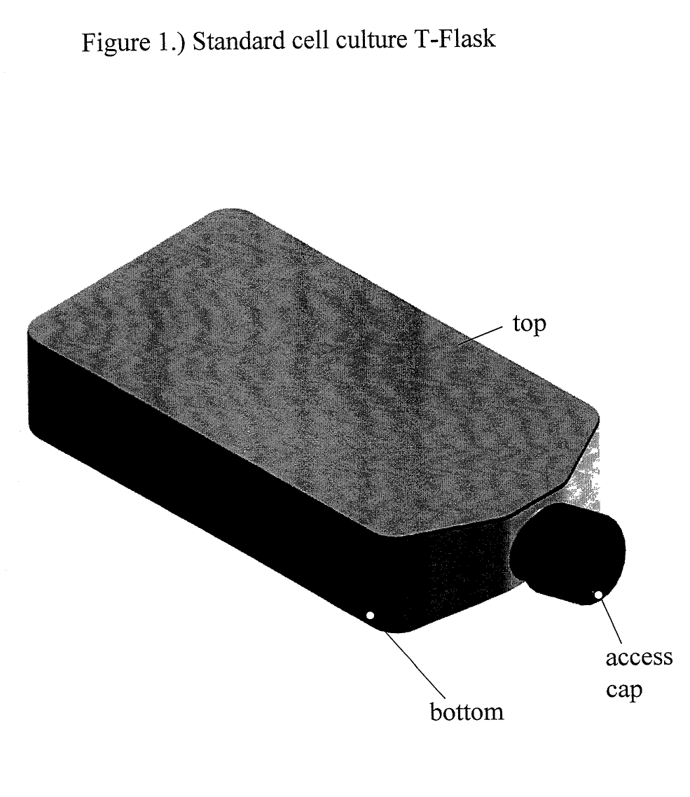

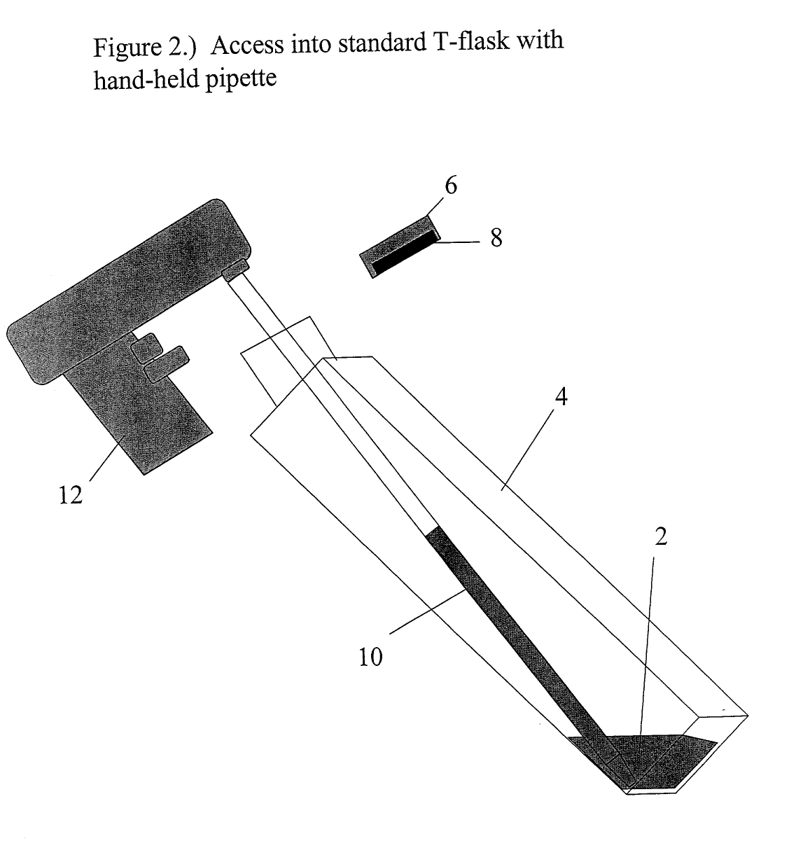

### DESCRIPTION OF THE INVENTION

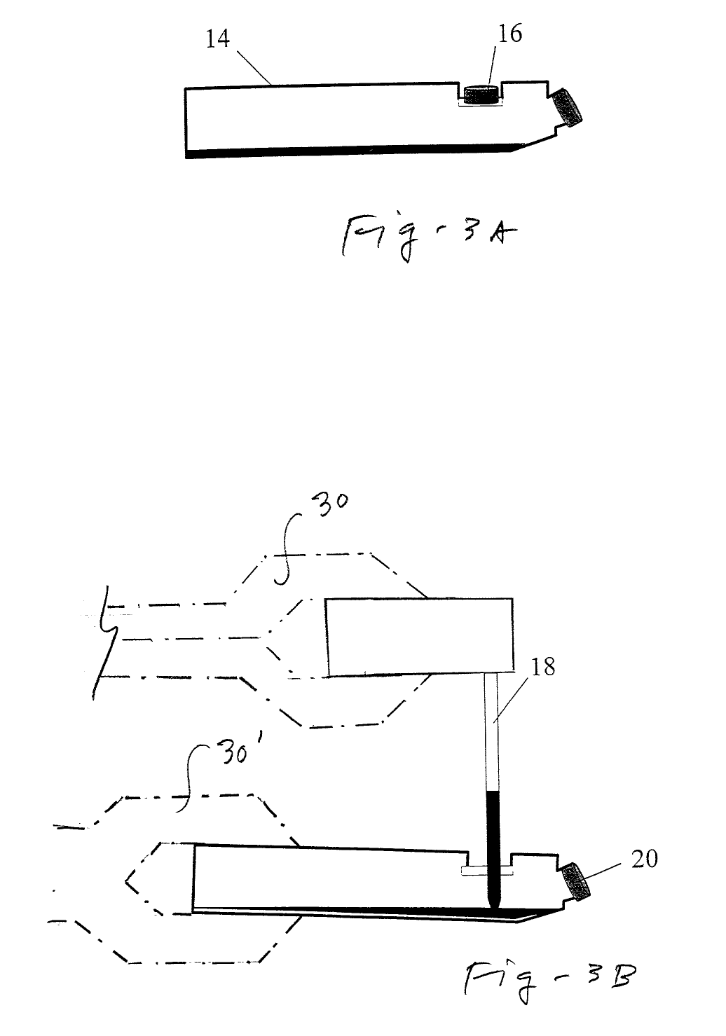

This invention is directed to a tissue culture flask vessel that provides an access port which is more amenable to an automated removal and delivery of fluid. FIG. 3A is a drawing that shows a preferred implementation of such a design. As shown, the design 14 uses the same basic T-flask, but also incorporates a lid 16 located on the top side of the flask. A slight tilt of the flask now locates the fluid in the front corner of the flask which can easily be accessed by a top-down pipette of much shorter length 18, on the order of two inches. This greatly reduces the angular tolerances for pipette access as well as significantly reduces the amount of tilt manipulation required to locate the fluid for pipette access.

All of these factors greatly increase the robustness of an automated approach to cell culture. In particular, FIG. 3B illustrates a robotic manipulation of the flask of FIG. 3A. In this case one or more multi-axis arms having appropriate end effectors 30 and 30′ are used to manipulate the pipette 18 and/or flask 14. Such arms are commercially available from a variety of manufacturers, including but not limited to Adept, Inc., CRS Robotics, FANUC Robotics, Motoman, Rixan Associates, Seiko D-Tran, and others.

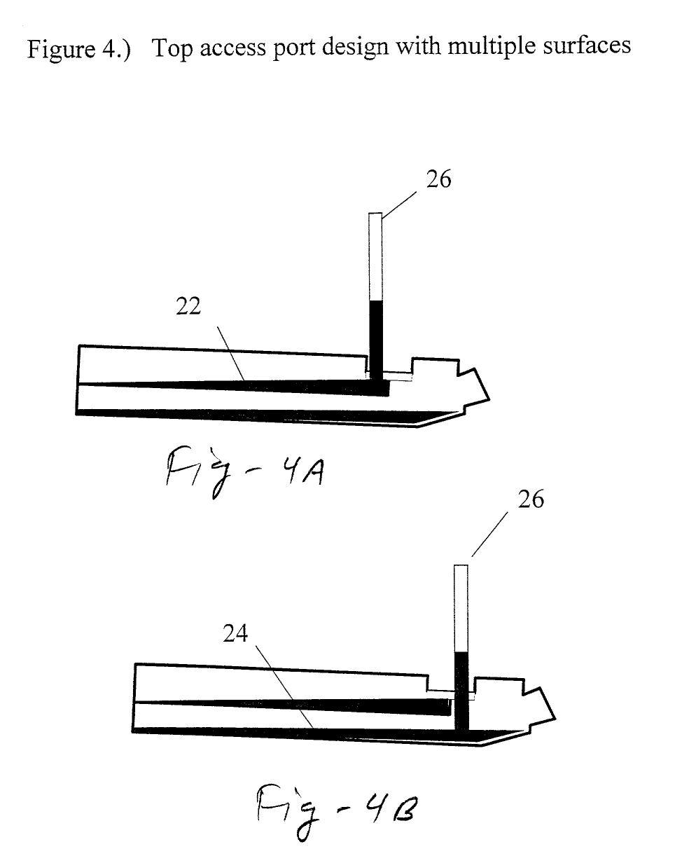

In the preferred embodiment and as shown in FIGS. 3 and 4, the lid access on flasks according to the invention is recessed so that the flask can also be stacked on top of each other, as is common practice in storage. It may be advantageous to preserve the existing lid 20 as found on current flasks designs to maintain proper gas exchange.

Another implementation may allow for a multiple layer flask which allows for two or more surfaces to be accessed within the same flask by a single top access port. This is accomplished with access to the upper and lower level surfaces using a short pipette [26]. FIG. 4A shows liquid being added to or drawn from the upper level 22, whereas FIG. 4B is a drawing of the two-level flask shown in FIG. 4A with liquid being added to or drawn from the lower level 24. It will be appreciated that although two levels are shown, three or more may be accommodated through appropriate engineering modification.

I claim:

1. An improved cell culture flask, comprising:
a flattened, fluid-holding vessel having top and bottom surfaces and an interior with a cell-culture surface; and
a port extending through the top surface providing direct access to the cell-culture surface, thereby limiting the required pipette length for introduction and/or removal of fluid.

2. The cell culture flask of claim 1, wherein the access port is recessed at or below the top surface enabling multiple flasks to be stacked on top of one another.

3. The cell culture flask of claim 1, wherein the access port includes a removable cap.

4. The cell culture flask of claim 1, wherein the access port further includes a membrane filter limiting the transmission of airborne contaminants.

5. The cell culture flask of claim 1, wherein the interior includes multiple cell-culture surfaces, each accessible through the port extending through the top surface.
